(19) United States
(12) Patent Application Publication
(10) Pub. No.: US 2015/0112107 A1
(43) Pub. Date: Apr. 23, 2015

(54) OXYGENATE-TO-OLEFINS PROCESS AND AN APPARATUS THEREFOR

(71) Applicant: SHELL OIL COMPANY, Houston, TX (US)

(72) Inventors: Rajaram Ramesh, Amsterdam (NL); Jeroen Van Westrenen, Amsterdam (NL)

(21) Appl. No.: 14/368,649

(22) PCT Filed: Dec. 21, 2012
(86) PCT No.: PCT/EP2012/076849

§ 371 Date: Jun. 25, 2014

(30) Foreign Application Priority Data
Dec. 28, 2011 (EP) 11195905.2

(51) Int. Cl. C07C 7/11; C07C 7/00; C07C 1/22

(52) U.S. Cl. CPC C07C 7/11; C07C 1/22; C07C 7/005; C07C 2529/40
USPC 585/324; 422/187

(57) ABSTRACT

The present invention relates to a process for the preparation of an olefinic product, such as one or both of ethylene and propylene, from an oxygenate feedstock, such as methanol, and an apparatus therefore, said process comprising: treating an effluent stream with a carbonyl compound absorbent stream comprising an aqueous solution of bisulphite having a pH in the range of from 4 to 8, to provide an olefinic product stream comprising olefin and a loaded carbonyl compound absorbent stream comprising an aqueous solution of at least one carbonyl adduct comprising one or both of C2+ aldehyde adduct and ketone adduct and optionally unreacted bisulphite, said liquid absorbent stream and loaded carbonyl compound absorbent stream in a carbonyl compound absorbent circuit separate from the effluent separation circuit.

### FIELD OF THE INVENTION

The present invention relates to a process for the preparation of an olefinic product, such as one or both of ethylene and propylene, from an oxygenate feedstock, such as methanol, and an apparatus therefor.

### BACKGROUND OF THE INVENTION

Conventionally, ethylene and propylene are produced via steam cracking of paraffinic feedstocks including ethane, propane, naphtha and hydrowax. An alternative route to ethylene and propylene is an oxygenate-to-olefin (OTO) process. Interest in OTO processes for producing ethylene and propylene is growing in view of the increasing availability of natural gas. Methane in the natural gas can be converted into for instance methanol or dimethylether (DME), both of which are suitable feedstocks for an OTO process.

In an OTO process, an oxygenate such as methanol or dimethylether is provided to a reaction zone of a reactor comprising a suitable conversion catalyst and converted to ethylene and propylene. In addition to the desired ethylene and propylene, a substantial part of the oxygenate such as methanol is converted to higher hydrocarbons including C4+ olefins, paraffins and carbonaceous deposits on the catalyst. The effluent from the reactor comprising the olefins, any unreacted oxygenates such as methanol and dimethylether and other reaction products such as water may then be treated to provide separate component streams. Unreacted oxygenates can be separated from the reaction effluent, for instance by contacting with a cooled aqueous stream in a quench tower.

In order to increase the ethylene and propylene yield of the process, the C4+ olefins may be recycled to the reaction zone or alternatively further cracked in a dedicated olefin cracking zone to produce further ethylene and propylene.

Due to the high temperatures in the reaction zone and the acidity of the catalyst, a portion of the oxygenates such as methanol may unavoidably decompose thermally or catalytically into oxides of carbon, i.e. carbon monoxide and carbon dioxide in the gaseous form. The carbonaceous deposits on the catalyst can be removed by the periodic regeneration of the catalyst by heating it with an oxidising gas such as oxygen, in order to burn off the deposits.

Carbon dioxide generated during the OTO process is an acid gas which is thus present in the effluent from the reactor. In order to prevent contamination of the olefinic product and problems associated with the formation of solid carbon dioxide during the separation of the olefinic product into olefinic component streams, which may be carried out at cryogenic temperatures, carbon dioxide should be removed from the reaction effluent and from the gaseous effluent from the quench tower before separation into olefinic component streams, for instance by treating with a caustic solution.

Carbonyl compounds, such as aldehydes and ketones, particularly formaldehyde and acetaldehyde, are commonly generated by the catalyst in side reactions and are also found in the effluent from the reactor. Carbonyl compounds may build up in the caustic solution used to remove carbon dioxide and other acid gases. The basic components of the caustic solution, such as hydroxide ions, can catalyse the aldol condensation and subsequent dehydration reactions of particularly acetaldehyde to form unsaturated aldehydes such as acrolein, especially at higher pH, such as a pH of greater than 9. Unsaturated aldehydes may polymerise when allowed to accumulate in the caustic solution and if the aldol condensation reaction is left unchecked, a viscous oily polymer can be formed, known as ‘red oil’, which is insoluble in the caustic solution and can deposit on equipment internals, causing fouling.

WO 2007/111744 discloses a process for oxygenate conversion to olefins with enhanced carbonyl recovery. A recycle or circulated water stream is treated with a sulphite-containing material in order to form a treated water stream with an appropriately reduced or minimised carbonyl, and particularly aldehyde, content. The sulphite-containing material is added to the oxygenate absorber zone. The oxygenate-rich water stream containing unreacted sulphite and bisulphite addition compounds produced in the oxygenate absorber zone is passed to an oxygenate stripper zone to be separated into an oxygenate-containing stream and a recycle water stream. The oxygenate-containing stream can be returned to the oxygenate conversion reactor. The recycle water stream can be passed to a wash water stripper to recover oxygenates and provide a bottoms water stream comprising unreacted sulphite and bisulphite addition compounds which can be passed to the effluent treatment zone for the treatment of the reactor section effluent. The recycle water stream can also be passed to the oxygenate absorber zone for the treatment of the compressed oxygenate conversion effluent stream.

### SUMMARY OF THE INVENTION

The present invention addresses the problem of red oil formation in an OTO process by treating the effluent from the reactor, after the separation of unreacted oxygenates from the reaction stream and subsequent compression, with a carbonyl compound absorbent stream comprising an aqueous solution of bisulphite. The bisulphite in the carbonyl compound absorbent stream forms a water soluble adduct, particularly a hydroxyalkyl sulfonate salt, with any carbonyl compounds present, particularly one or both of aldehydes and ketones, thereby removing them from the gaseous effluent from the reactor into the aqueous solution and lowering the resulting concentration of dissolved aldehyde and ketones in the aqueous solution. In this way, the fouling of the caustic solution by the formation of red oil from the reaction of carbonyl compounds is mitigated.

In the process of WO 2007/111744, sulphite-containing material is added to the oxygenate absorber zone. The integration of the water treatment system means that sulphite-containing material is distributed in the water streams provided to the effluent treatment zone, oxygenate absorber zone, oxygenate stripper zone and wash water stripper.

Aldehydes such as formaldehyde, as well as ketones, are commonly present in the oxygenate conversion reactor effluent stream. Different aldehydes and ketones form adducts with bisulphite. The adduct forming reaction is reversible, such that an equilibrium between the aldehyde or ketone and bisulphite reactants and adduct product exists. Each aldehyde or ketone has its own equilibrium with bisulphite. Formaldehyde is most favoured to form an adduct with bisulphite, compared to the more sterically hindered C2+ aldehydes and ketones, such that it will displace C2+ aldehydes and ketones from an adduct of C2+ aldehyde or ketone and bisulphite present in the aqueous solution.

In the process of WO 2007/111744, formaldehyde present in the oxygenate conversion effluent stream can be absorbed in the effluent treatment zone. This absorption is carried out using the bottoms water stream from the wash water stripper. This bottoms water stream comprises unreacted sulphite and bisulphite addition compounds from the oxygenate absorber zone, particularly acetaldehyde adduct, leading to the potential release of C2+ aldehydes in the effluent treatment zone by the preferential formation of formaldehyde adduct, particularly when the bisulphite concentration is similar to the concentration of formaldehyde. Typically, more formaldehyde than acetaldehyde may be formed as a by-product in the OTO reaction, such that the displacement of acetaldehyde from its adduct by formaldehyde in the effluent treatment zone is likely, particularly if the bisulphite concentration is too low.

In contrast, in the present invention the water stream from the oxygenate absorber zone (the carbonyl compound absorption zone in the process described herein) is not passed to the effluent treatment zone. Instead, an aqueous liquid stream in an effluent treatment circuit independent from the carbonyl compound absorbent circuit comprising bisulphate is used to treat the reaction effluent which would be free of bisulphite and aldehyde adduct. Consequently, there could be no release of C2+ aldehyde or ketone from their adducts upon contact of the aqueous liquid stream with formaldehyde.

In addition, in the process of WO 2007/111744, the oxygenate-containing overhead stream from the oxygenate stripper zone can be recycled to the oxygenate conversion reactor section. The oxygenate-containing overhead stream may be contaminated with volatile aldehydes and/or ketones such as formaldehyde and acetaldehyde as well as oxides of sulphur. The bisulphite addition compounds of volatile aldehydes and/or ketones are thermally labile in aqueous solutions, such that distillation in the oxygenate stripper zone can cause the decomposition of the adduct into the aldehyde or ketone and bisulphite. Volatile aldehydes and ketones may vaporise from the solution, altering the equilibrium between reactant and adduct in favour of the reactants, thereby promoting further decomposition of the bisulphite addition compounds. Volatile aldehyde or ketone would exit the oxygenate stripper zone in the oxygenate-containing overhead stream and could be returned to the oxygenate conversion reactor section.

Furthermore, if the pH of the sulphite and bisulphite solutions are not maintained above 4, sulphur dioxide, SO₂, may be formed. At the oxygenate stripping temperatures, sulphur dioxide may be vaporised from the solution and exit the stripping zone in the oxygenate-containing overhead stream which can be returned to the oxygenate conversion reactor section. Sulphur dioxide is corrosive and may degrade the supply lines to and from the reactor, as well as the reactor section itself. In addition, the effluent from the reactor may be contaminated with sulphur compounds, which is undesirable as this may require additional processing of the effluent. Sulphur may also be present in the carbonaceous deposits formed on the OTO catalyst.

The oxidative regeneration of an oxygenate conversion catalyst having absorbed sulphur-comprising compounds to remove carbonaceous deposits will result in the oxidation of the sulphur-comprising compounds, producing oxides of sulphur, SOx, such as sulphur dioxide and sulphur trioxide in the regeneration effluent. Oxides of sulphur are air borne pollutants which contaminate the regeneration effluent stream. Treatment of the regeneration effluent may therefore be required to remove these oxides of sulphur.

In contrast, the present invention seeks to mitigate against the contamination of the oxygenate to olefin catalyst with contaminants such as sulphur compounds like SO₂. This is achieved by providing the carbonyl compound absorbent comprising bisulphite in a separate circuit to that of the aqueous liquid absorbent used to absorb oxygenate from the reaction effluent. In this way, sulphur compounds from the carbonyl compound absorbent are prevented from being passed to the reaction zone.

The process of the invention treats a compressed effluent stream derived from the conversion of an oxygenated feed stock to an olefinic product with a carbonyl compound absorbent stream comprising an aqueous solution of bisulphite having a pH in the range of from 4 to 8. Bisulphite in this range of pH can form a water soluble adduct with any carbonyl compounds present, particularly aldehydes and ketones, thereby removing them from the effluent stream. This treatment can reduce the formation of aldol condensation products of the carbonyl compounds, reducing the formation of red oil and the associated fouling of equipment internals downstream of the treatment point. This is particularly beneficial in preventing red oil formation in the aqueous alkaline solution used to remove any acid gas from the effluent stream downstream of the treatment point.

In addition, the process of the present invention prevents transfer of one or more of the carbonyl compound absorbent, the loaded carbonyl compound absorbent comprising an aqueous solution of the water soluble adduct, the decomposition products of the loaded carbonyl compound absorbent and sulphur compounds such as sulphur dioxide to the aqueous liquid used to treat, typically quench, the reaction effluent stream to remove water, oxygenate and formaldehyde. This prevents contamination of the aqueous liquid or any recovered oxygenate with components or derivatives of the carbonyl compound absorbent thus avoiding the potential release of C2+ aldehyde or ketone upon contact with formaldehyde in the reaction effluent stream and the contamination of the oxygenate catalyst with sulphur-comprising compounds.

In a first aspect, the present invention provides a process for the preparation of olefinic product, the process comprising at least the steps of:
reacting an oxygenate feedstock comprising oxygenate in an oxygenate reaction zone in the presence of a catalyst comprising a molecular sieve to produce a reaction effluent stream comprising oxygenate, olefin, water and carbonyl compound comprising formaldehyde and one or both of C2+ aldehyde and ketone;
treating the reaction effluent stream with an aqueous liquid stream to provide a water rich stream comprising oxygenate, formaldehyde and water and a water depleted effluent stream comprising olefin and carbonyl compound comprising one or both of C2+ aldehyde and ketone, said aqueous liquid stream and said water rich stream present in an effluent separation circuit;
compressing the water depleted effluent stream, with the optional removal of any condensed phase, to provide a compressed effluent stream;
treating the compressed effluent stream with a carbonyl compound absorbent stream comprising an aqueous solution of bisulphite having a pH in the range of from 4 to 8, to provide an olefinic product stream comprising olefin and a loaded carbonyl compound absorbent stream comprising an aqueous solution of at least one carbonyl compound adduct comprising one or both of C2+ aldehyde adduct and ketone adduct and optionally unreacted bisulphite, said carbonyl compound absorbent stream and loaded carbonyl compound absorbent stream in a carbonyl compound absorbent circuit separate from the effluent separation circuit.

The bisulphite treatment should occur before any acid gas absorption treatment, such as contact with an aqueous alkaline stream, or as the first step in such an acid gas absorption treatment.

As used herein, the term “Cn+” represents a compound having n or more carbon atoms. For instance, a C2+ aldehyde represents and aldehyde having 2 or more carbon atoms i.e. those aldehydes excluding formaldehyde.

In the present context, the term “separate” used in relation to the carbonyl compound absorbent and effluent separation circuits, means that there is no transfer of the carbonyl compound absorbent and/or the loaded carbonyl compound absorbent to the effluent separation circuit. This prevents the contamination of the aqueous liquid in the effluent separation circuit with bisulphite and/or carbonyl compound adduct. It will therefore be apparent that the aqueous liquid stream and water rich stream should not comprise an aqueous solution of bisulphite having a pH in the range of from 4 to 8 and/or carbonyl compound adduct comprising one or both of C2+ aldehyde adduct and ketone adduct.

Avoiding such a contamination of the effluent separation circuit will therefore prevent contamination of any recovered oxygenate, thereby allowing it to be recycled and released again into the reaction quench zone or to be recycled to the oxygenate reaction zone without contaminating the catalyst with the sulphur-comprising compounds of the carbonyl compound absorbent. This can minimise the presence of oxides of sulphur in the regeneration effluent stream produced by the oxidative regeneration of the deactivated catalyst.

A pH of below 4 can cause the decomposition of the bisulphite solution to liberate SO₂, which can cause corrosion to occur. As pH increases above 6, conversion of bisulphite to sulphite occurs, such that at a pH above 8, the bisulphite has almost entirely been converted to sulphite. A preferred pH for the bisulphite solution is in the range of from 4.5 to 7, still more preferably in the range of from 5 to 6.5. The cationic counter-ion to the bisulphite anion may be one of those commonly known in the art, such as a cation selected from the group comprising the alkali metal ions and alkaline earth metal ions, particularly lithium, sodium and potassium, with the sodium cation being preferred. The bisulphite solution may have a concentration of bisulphite, such as sodium bisulphite, in the range of from 1 to 10 wt. %, more preferably 1 to 5 wt. %, based on the bisulphite solution.

Preferably, the compressed effluent stream is treated with a carbonyl compound absorbent stream comprising an aqueous solution of bisulphite having a pH in the range of from 4 to 8 at a temperature in the range of from 30 to 50° C., more preferably of from 35 to 45° C.

In one embodiment, the process further comprises the steps of:
separating at least a part of the water rich stream into an oxygenate recovered stream comprising oxygenate and an aqueous recovered stream comprising water;
passing the oxygenate recovered stream to the oxygenate reaction zone.

In another embodiment, the process further comprises the step of:
passing at least a part of the aqueous recovered stream to the aqueous liquid stream.

In a further embodiment, the process further comprises the step of:
passing at least a part of the water rich stream to the aqueous liquid stream.

In another embodiment, the process further comprises the step of:
passing at least a part of the loaded carbonyl compound absorbent stream to the carbonyl compound absorbent stream.

In yet another embodiment, the process further comprises the steps of:
removing at least a portion of the loaded carbonyl compound absorbent stream as a continuing loaded carbonyl compound absorbent stream; and
adding carbonyl compound absorbent comprising an aqueous solution of bisulphite having a pH in the range of from 4 to 8 to the carbonyl compound absorbent stream as a carbonyl compound absorbent restoration stream.

In another embodiment, the C2+ aldehyde may comprise one or both of a saturated C2+ aldehyde, such as acetaldehyde and an unsaturated C3+ aldehyde, such as acrolein.

In a still further embodiment, the reaction effluent stream, water depleted stream, compressed effluent stream and olefinic product stream each further comprise acid gas, such as one or both of carbon dioxide and hydrogen sulphide.

In another embodiment of the process, the pH of the aqueous solution of bisulphite and/or the carbonyl compound absorbent stream may be independently adjusted in the range of from 5 to 8, more preferably of from 5 to 7. Maintaining the pH in these ranges allows the at least partial absorption of any carbon dioxide present in the compressed effluent stream by the carbonyl compound absorbent stream. The acidic carbon dioxide can be converted into bicarbonate by the carbonyl compound absorbent stream at higher pH, acting as a buffer to maintain the stream at a pH of at or below 9.

In a still further embodiment, the process may further comprise the steps of:
treating the olefinic product stream with an acid gas absorbent stream to provide a loaded acid gas absorbent stream and an acid gas depleted olefinic product stream comprising olefin.

The treating of the olefinic product stream with an acid gas absorbent stream may comprise treatment with a plurality of acid gas absorbent streams.

In another embodiment, the step of treating the olefinic product stream comprises:
contacting the olefinic product stream with an acid gas absorbent stream comprising an aqueous alkaline solution to provide a loaded acid gas absorbent stream comprising a loaded alkaline aqueous solution and the acid gas depleted olefinic product stream.

The aqueous alkaline solution, such as aqueous sodium hydroxide, may have a concentration in the range of from 1 to 10 wt. %, more preferably 1 to 6 wt. %, based on the aqueous alkaline solution.

In one embodiment, the olefinic product stream can be contacted with a plurality of acid gas absorbent streams, each stream comprising aqueous alkaline solution at a different concentration (in wt. %). Typically, the plurality of aqueous alkaline solutions may comprise an increasing concentration of aqueous alkali with each contacting.

The ability of the carbonyl compound absorbent stream to absorb at least a portion of any acid gas, such as carbon dioxide, is beneficial because it provides the partial absorption of the acid gas prior to treatment of the olefinic product stream with an acid gas absorbent stream.

In a further embodiment, treatment of the olefinic product stream with an acid gas absorbent stream can be followed by treatment, typically contacting, with an aqueous stream to provide the acid gas depleted olefinic product stream. The treatment with an aqueous stream can remove any entrained components of the acid gas absorbent stream, such as the aqueous alkaline solution.

In another embodiment, the acid gas depleted olefinic product stream may comprise two or more of the group selected from ethylene, propylene, butylenes, pentylenes and hexylenes. In yet another embodiment, the process may further comprise the steps of:
drying and optionally compressing the acid gas depleted olefinic product stream to provide a dried acid gas depleted olefinic product streams;
separating the dried acid gas depleted olefinic product stream into two or more olefinic component streams, each said olefinic component stream comprising at least one of the group selected from ethylene, propylene, butylenes, pentylenes and hexylenes.

In another embodiment of the process, the oxygenate feedstock may be reacted to produce the reaction effluent stream in the presence of an olefinic co-feed, such as an olefinic co-feed comprising one or both of butylene and pentylene.

In a still further embodiment, the molecular sieve may be selected from the group comprising silicoaluminophosphate and aluminosilicate. The molecular sieve may be preferably an aluminosilicate having at least a 10-membered ring zeolite structure. Still more preferably, the aluminosilicate may comprise one or more of the group comprising a TON-type aluminosilicate, such as ZSM-22, a MTT-type aluminosilicate, such as ZSM-23, MEL-type aluminosilicate, such as ZSM-11 and MFI-type aluminosilicate, such as ZSM-5.

In a second aspect, the present invention provides an apparatus for the preparation of an olefinic product, from an oxygenate feedstock, said apparatus comprising at least:
an oxygenate reaction zone comprising a catalyst comprising molecular sieve, said oxygenate reaction zone having a first inlet for an oxygenate feedstock stream comprising oxygenate and a first outlet for a reaction effluent stream comprising oxygenate, olefin, water and carbonyl compound comprising formaldehyde and one or both of C2+ aldehyde and ketone, said first outlet in fluid communication with a first inlet of an effluent separation zone;
an effluent separation zone for separating the reaction effluent stream into a water rich stream comprising oxygenate, formaldehyde and water and a water depleted effluent stream comprising olefin and carbonyl compound comprising one or both of C2+ aldehyde and ketone, said effluent separation zone having a first inlet for the reaction effluent stream, a second inlet for an aqueous liquid stream, a first outlet for the water rich stream and a second outlet for the water depleted effluent stream, said second outlet in fluid communication with the inlet of an effluent compressor, wherein said aqueous liquid stream and said water rich stream comprise an effluent separation circuit;
an effluent compressor having a first inlet for the water depleted effluent stream and a first outlet for a compressed effluent stream, said first outlet in fluid communication with the first inlet of an carbonyl compound absorption zone, said effluent compressor optionally comprising gas/liquid separation means for the removal of any condensed phase;
a carbonyl compound absorption zone having a first inlet for the compressed effluent stream, a second inlet for a carbonyl compound absorbent stream comprising an aqueous solution of bisulphite having a pH in the range of from 4 to 8, a first outlet for an olefinic product stream comprising olefin and a second outlet for a loaded carbonyl compound absorbent stream comprising an aqueous solution of at least one carbonyl compound adduct comprising one or both of C2+ aldehyde adduct and ketone adduct and optionally unreacted bisulphite, wherein said carbonyl compound absorbent stream and said loaded carbonyl compound absorbent stream comprise a carbonyl compound absorbent circuit, said carbonyl compound absorbent circuit being separate from the effluent separation circuit.

In one embodiment of the second aspect, the apparatus may further comprise:
an acid gas absorption zone to separate acid gas from the olefinic product stream to provide an acid gas depleted olefinic product stream, said acid gas absorption zone having a first inlet for the olefinic product stream in fluid communication with the first outlet of the carbonyl compound absorption zone, a second inlet for an acid gas absorbent stream, for instance comprising an aqueous alkaline solution, a first outlet for the acid gas depleted olefinic product stream and a second outlet for a loaded acid gas absorbent stream, for instance comprising a loaded alkaline aqueous solution.

In another embodiment of the second aspect, the apparatus may further comprise:
an olefinic product compressor having a first inlet for the acid gas depleted olefinic product stream in fluid communication with the first outlet of the acid gas absorption zone and a first outlet for a compressed acid gas depleted olefinic product stream in fluid communication with a first inlet of an olefinic separation zone, said olefinic product compressor optionally comprising gas/liquid separation means for the removal of any condensed phase;
an olefinic separation zone to separate the compressed acid gas depleted olefinic product stream into two or more olefinic component streams, said olefinic separation zone having a first inlet for the compressed acid gas depleted olefinic product stream, a first outlet for a first olefinic component stream and a second outlet for a second olefinic component stream.

In yet another embodiment of the second aspect, the apparatus may further comprise:
an oxygenate recovery zone to separate at least a part of the water rich stream into a oxygenate recovered stream comprising oxygenate and an aqueous recovered stream comprising water, said oxygenate recovery zone having a first inlet for the water rich stream in fluid communication with the first outlet of the effluent separation zone, a first outlet for the oxygenate recovered stream in fluid communication with an inlet of the oxygenate reaction zone and a second outlet for the aqueous recovered stream.

In a further embodiment of the second aspect, the apparatus may further comprise:
an oxygenate recovered stream heating furnace having a first inlet for the oxygenate recovered stream in fluid communication with the first outlet of the oxygenate recovery zone and a first outlet for a heated oxygenate recovered stream in fluid communication with an inlet of the oxygenate reaction zone.

### BRIEF DESCRIPTION OF THE DRAWINGS

FIG. 1 is a diagrammatic scheme of a process and apparatus for the preparation of an olefinic product described herein.

FIG. 2 is a diagrammatic scheme of another embodiment of a process and apparatus for the preparation of an olefinic product described herein.

FIG. 3 is a diagrammatic scheme of another embodiment of a process and apparatus for the preparation of an olefinic product described herein.

### DETAILED DESCRIPTION OF THE INVENTION

Embodiments of the present invention will now be described by way of example only and with reference to the accompanying non-limited drawing in which:

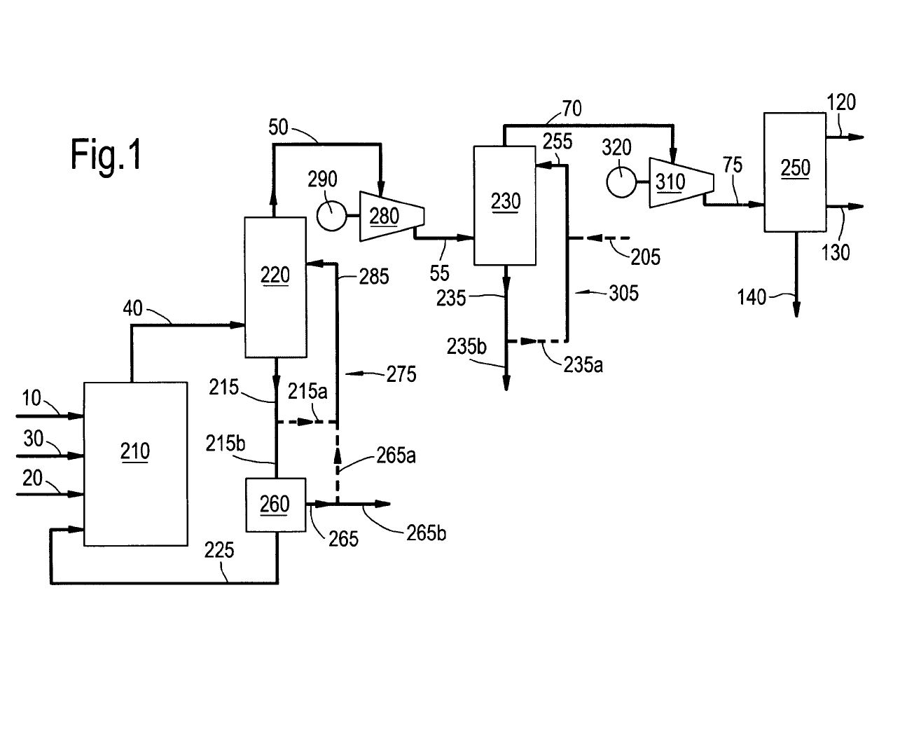

FIG. 1 is a diagrammatic scheme of a process and apparatus for the preparation of an olefinic product described herein.

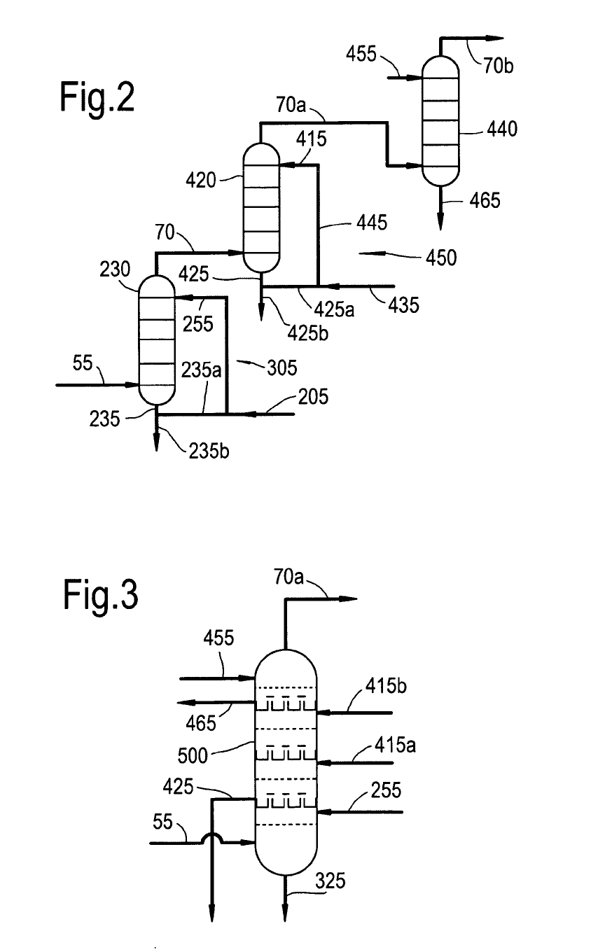

FIG. 2 is a diagrammatic scheme of another embodiment of a process and apparatus for the preparation of an olefinic product described herein.

FIG. 3 is a diagrammatic scheme of another embodiment of a process and apparatus for the preparation of an olefinic product described herein.

The process and apparatus described herein relates to a process for the catalytic conversion of an oxygenate feedstock to olefinic products in an oxygenate-to-olefin (OTO) process and the subsequent treatment of the reaction effluent from the reaction zone to remove carbonyl compound(s) comprising one or both of C2+ aldehyde and ketone.

FIG. 1 shows an apparatus 1 of one embodiment of the process described herein. An oxygenate feedstock stream 10 can be contacted in an oxygenate (or OTO) reaction zone 210, such as an OTO reactor, with a catalyst for oxygenate conversion under oxygenate conversion conditions, to obtain a reaction effluent comprising olefins, particularly lower olefins. The reaction effluent can be removed from reaction zone 210 as reaction effluent stream 40. Reaction effluent stream 40 may comprise unreacted oxygenate, olefin and water.

Reference herein to an oxygenate feedstock is to an oxygenate-comprising feedstock. In the OTO reaction zone 210, at least part of the feedstock is converted into a product containing one or more olefins, preferably including lower olefins, in particular ethylene and typically propylene.

The oxygenate used in the process is preferably an oxygenate which comprises at least one oxygen-bonded alkyl group. The alkyl group preferably is a C1-C5 alkyl group, more preferably C1-C4 alkyl group, i.e. comprises 1 to 5, or 1 to 4 carbon atoms respectively; more preferably the alkyl group comprises 1 or 2 carbon atoms and most preferably one carbon atom. Examples of oxygenates that can be used in the oxygenate feedstock include alcohols and ethers. Examples of preferred oxygenates include alcohols, such as methanol, ethanol, propanol; and dialkyl ethers, such as dimethyl ether, diethyl ether, methylethyl ether. Preferably, the oxygenate is methanol or dimethyl ether, or a mixture thereof.

Preferably the oxygenate feedstock comprises at least 50 wt. % of oxygenate, in particular methanol and/or dimethyl ether, based on total hydrocarbons, more preferably at least 70 wt. %.

An oxygenate co-feed may also be supplied by oxygenate recovered stream 225 discussed below.

A diluent, such as water or steam, may also be provided to the OTO reaction zone 210. In the embodiment of FIG. 1, the diluent is provided as diluent stream 30. The molar ratio of oxygenate to diluent may be between 10:1 and 1:10, preferably between 4:1 and 1:2, in particular when the oxygenate is methanol and the diluent is water (typically steam).

Preferably, in addition to the oxygenate and diluent, an olefinic co-feed is provided along with and/or as part of the oxygenate feedstock. FIG. 1 shows the co-feed being supplied to OTO reaction zone 210 as an olefinic co-feed stream 20. Reference herein to an olefinic co-feed is to an olefin-comprising co-feed.

The olefinic co-feed preferably comprises C4+ olefins i.e. C4 and higher olefins, more preferably C4 and C5 olefins. Preferably, the olefinic co-feed comprises at least 25 wt. %, more preferably at least 50 wt. %, of C4 olefins, and at least a total of 70 wt. % of C4 hydrocarbon species.

In order to maximize production of ethylene and propylene, it is desirable to maximize the recycle of C4 olefins in the effluent of the OTO process. This can be done by recycling at least part of the C4+ hydrocarbon fraction, preferably C4-C5 hydrocarbon fraction, more preferably C4 hydrocarbon fraction, in the OTO effluent. However, a certain part thereof, such as between 1 and 5 wt. %, can be withdrawn as purge, since otherwise saturated hydrocarbons, in particular C4s (normal and iso butane) may build up in the process, which are substantially not converted under the OTO reaction conditions. Preferably, at least 70 wt. % of the olefinic co-feed, during normal operation, is formed by a recycle stream of a C4+ hydrocarbon fraction from the OTO reaction effluent. Preferably at least 90 wt. % of olefinic co-feed, based on the whole olefinic co-feed, is formed by such recycle stream.

The preferred molar ratio of oxygenate in the oxygenate feedstock to olefin in the olefinic co-feed provided to the OTO conversion zone 210 depends on the specific oxygenate used and the number of reactive oxygen-bonded alkyl groups therein. Preferably the molar ratio of oxygenate to olefin in the total feed lies in the range of 20:1 to 1:10, more preferably in the range of 18:1 to 1:5, still more preferably in the range of 15:1 to 1:3, even still more preferably in the range of 12:1 to 1:3.

A variety of OTO processes are known for converting oxygenates, such as for instance methanol or dimethyl ether to an olefin-containing product, as already referred to above. One such process is described in WO A 2006/020083. Processes integrating the production of oxygenates from synthesis gas and their conversion to light olefins are described in US20070203380A1 and US20070155999A1.

Catalysts suitable for converting the oxygenate feedstock comprise molecular sieve. Such molecular sieve-comprising catalysts typically also include binder materials, matrix material and optionally fillers. Suitable matrix materials include clays, such as kaolin. Suitable binder materials include silica, alumina, silica-alumina, titania and zirconia, wherein silica is preferred due to its low acidity.

Molecular sieves preferably have a molecular framework of one, preferably two or more corner-sharing tetrahedral units, more preferably, two or more [SiO4], [A1O4] and/or [PO4] tetrahedral units. These silicon, aluminium and/or phosphorus based molecular sieves and metal containing silicon, aluminium and/or phosphorus based molecular sieves have been described in detail in numerous publications including for example, U.S. Pat. No. 4,567,029. In a preferred embodiment, the molecular sieves have 8-, 10- or 12-ring structures and an average pore size in the range of from about 3 Å to 15 Å. Suitable molecular sieves are silicoaluminophosphates (SAPO), such as SAPO-17, -18, 34, -35, -44, but also SAPO-5, -8, -11, -20, -31, -36, 37, -40, -41, -42, -47 and -56; aluminophosphates (AlPO) and metal substituted (silico)aluminophosphates (MeAlPO), wherein the Me in MeAlPO refers to a substituted metal atom, including metal selected from one of Group IA, IIA, IB, IIIB, IVB, VB, VIIB, VIIB, VIIIB and Lanthanides of the Periodic Table of Elements. Preferably Me is selected from one of the group consisting of Co, Cr, Cu, Fe, Ga, Ge, Mg, Mn, Ni, Sn, Ti, Zn and Zr.

Alternatively, the conversion of the oxygenate feedstock may be accomplished by the use of an aluminosilicate-comprising catalyst, in particular a zeolite-comprising catalyst. Suitable catalysts include those containing a zeolite of the ZSM group, in particular of the MFI type, such as ZSM-5, the MTT type, such as ZSM-23, the TON type, such as ZSM-22, the MEL type, such as ZSM-11, and the FER type. Other suitable zeolites are for example zeolites of the STF-type, such as SSZ-35, the SFF type, such as SSZ-44 and the EU-2 type, such as ZSM-48.

Aluminosilicate-comprising catalyst, and in particular zeolite-comprising catalyst are preferred when an olefinic co-feed is fed to the oxygenate conversion zone together with oxygenate, for increased production of ethylene and propylene.

Preferred catalysts comprise a more-dimensional zeolite, in particular of the MFI type, more in particular ZSM-5, or of the MEL type, such as zeolite ZSM-11. Such zeolites are particularly suitable for converting olefins, including iso-olefins, to ethylene and/or propylene. The zeolite having more-dimensional channels has intersecting channels in at least two directions. So, for example, the channel structure is formed of substantially parallel channels in a first direction, and substantially parallel channels in a second direction, wherein channels in the first and second directions intersect. Intersections with a further channel type are also possible. Preferably, the channels in at least one of the directions are 10-membered ring channels. A preferred MFI-type zeolite has a silica-to-alumina ratio, SAR, of at least 60, preferably at least 80. More preferred MFI-type zeolites have a silica-to-alumina ratio in the range of from 60 to 150, more preferably of from 80 to 100,

Particular catalysts include catalysts comprising one or more zeolites having one-dimensional 10-membered ring channels, i.e. one-dimensional 10-membered ring channels, which are not intersected by other channels. Preferred examples are zeolites of the MTT and/or TON type. Preferably, the catalyst comprises at least 40 wt. %, preferably at least 50% wt. of such zeolites based on total zeolites in the catalyst. In one embodiment, the catalyst comprises in addition to one or more one-dimensional zeolites having 10-membered ring channels, such as of the MTT and/or TON type, a more-dimensional zeolite, in particular of the MFI type, more in particular ZSM-5, or of the MEL type, such as zeolite ZSM-11.

The catalyst may further comprise phosphorus as such or in a compound, i.e. phosphorus other than any phosphorus included in the framework of the molecular sieve. It is preferred that a MEL or MFI-type zeolite comprising catalyst additionally comprises phosphorus. The phosphorus may be introduced by pre-treating the MEL or MFI-type zeolites prior to formulating the catalyst and/or by post-treating the formulated catalyst comprising the MEL or MFI-type zeolites. Preferably, the catalyst comprising MEL or MFI-type zeolites comprises phosphorus as such or in a compound in an elemental amount of from 0.05 to 10 wt. % based on the weight of the formulated catalyst. A particularly preferred catalyst comprises phosphorus and MEL or MFI-type zeolite having SAR of in the range of from 60 to 150, more preferably of from 80 to 100. An even more particularly preferred catalyst comprises phosphorus and ZSM-5 having SAR of in the range of from 60 to 150, more preferably of from 80 to 100.

It is preferred that molecular sieves in the hydrogen form are used in the oxygenate conversion catalyst, e.g., HZSM-22, HZSM-23, and HZSM-48, HZSM-5. Preferably at least 50% w/w, more preferably at least 90% w/w, still more preferably at least 95% w/w and most preferably 100% of the total amount of molecular sieve used is in the hydrogen form. It is well known in the art how to produce such molecular sieves in the hydrogen form. The reaction conditions of the oxygenate conversion, include a reaction temperature of 350 to 1000° C., preferably from 350 to 750° C., more preferably 450 to 700° C., even more preferably 500 to 650° C.; and a pressure from 0.1 kPa (1 mbar) to 5 MPa (50 bar), preferably from 100 kPa (1 bar) to 1.5 MPa (15 bar).

Preferably, the oxygenate feedstock is preheated to a temperature in the range of from 200 to 550° C., more preferably 250 to 500° C. prior to contacting with the molecular sieve-comprising catalyst.

The catalyst particles used in the process can have any shape known to the skilled person to be suitable for this purpose, and can be present in the form of spray dried catalyst particles, spheres, tablets, rings, extrudates, etc. Extruded catalysts can be applied in various shapes, such as, cylinders and trilobes. Spray-dried particles allowing use in a fluidized bed or riser reactor system are preferred. Spherical particles are normally obtained by spray drying. Preferably the average particle size is in the range of 1-200 μm, preferably 50-100 μm.

Although the C4+ hydrocarbon fraction in the reaction effluent may be recycled as an olefinic co-feed as discussed above, in an alternative embodiment not shown in FIG. 1, at least part of the olefins in the C4+ hydrocarbon fraction are converted to ethylene and/or propylene by contacting the C4+ hydrocarbon fraction in a separate unit with a molecular sieve-comprising catalyst, particularly a zeolite-comprising catalyst. This is particularly preferred where molecular sieve-comprising catalyst in the OTO process comprises a least one SAPO, ALPO, or MeAlPO type molecular sieve, preferably SAPO-34. These catalysts are less suitable for converting olefins. Preferably, the C4+ hydrocarbon fraction, such as the third olefinic component stream 140 described below, is contacted with the zeolite-comprising catalyst at a reaction temperature of 350 to 1000° C., preferably from 375 to 750° C., more preferably 450 to 700° C., even more preferably 500 to 650° C.; and a pressure from 0.1 kPa (1 mbar) to 5 MPa (50 bar), preferably from 100 kPa (1 bar) to 1.5 MPa (15 bar).

Optionally, the stream comprising C4+ olefins also contains a diluent. Examples of suitable diluents include, but are not limited to, liquid water or steam, nitrogen, argon, paraffins and methane. Under these conditions, at least part of the olefins in the C4+ hydrocarbon fraction are converted to further ethylene and/or propylene. The further ethylene and/or propylene may be combined with the further ethylene and/or propylene obtained directly from the OTO reaction zone 210. Such a separate process step directed at converting C4+ olefins to ethylene and propylene is also referred to as an olefin cracking process (OCP).

Catalysts comprising molecular sieve, particularly aluminosilicate-comprising catalysts, and more particularly zeolite-comprising catalysts, have the further advantage that in addition to the conversion of methanol or ethanol, these catalysts also induce the conversion of olefins to ethylene and/or propylene. Therefore, aluminosilicate-comprising catalysts, and in particular zeolite-comprising catalysts, are particularly suitable for use as the catalyst in an OCP. Particular preferred catalysts for the OCP reaction, i.e. converting part of the olefinic product, and preferably part of the C4+ hydrocarbon fraction of the olefinic product including C4+ olefins, are catalysts comprising at least one zeolite selected from MFI, MEL, TON and MTT type zeolites, more preferably at least one of ZSM-5, ZSM-11, ZSM-22 and ZSM-23 zeolites.

Both the OTO process and the OCP may be operated in a fluidized bed, e.g. a fast fluidized bed or a riser reactor system, and also in a fixed bed reactor, moving bed or a tubular reactor. A fluidized bed, e.g. a fast fluidized bed or a riser reactor system are preferred.

The catalyst can deactivate in the course of the OCP and OTO process. The deactivation occurs primarily due to deposition of carbonaceous deposits, such as coke, on the catalyst by side reactions. The deactivated catalyst can be regenerated to remove a portion of the carbonaceous deposit by methods known in the art. It is not necessary, and indeed may be undesirable, to remove all the carbonaceous deposit from the catalyst as it is believed that a small amount of residual carbonaceous deposit such as coke may enhance the catalyst performance. Additionally, it is believed that complete removal of the carbonaceous deposit may also lead to degradation of the molecular sieve.

The same catalyst may be used for both the OTO process and OCP. In such a situation, the catalyst comprising molecular sieve, particularly comprising aluminosilicate molecular sieve and more particularly comprising zeolite, may be first used in the OCP reaction zone for the conversion of the C4+ olefins of the C4+ hydrocarbon fraction. The catalyst from the OCP may then be used, typically without regeneration, in the OTO process for conversion of the oxygenate feedstock stream 10 and olefinic co-feed stream 20. The deactivated catalyst from the OTO process may then be regenerated as described herein, and the regenerated catalyst then used again in the OCP.

This line-up may be beneficial because it provides good heat integration between the OCP, OTO and regeneration processes. The OCP is endothermic and at least a portion of the heat of reaction can be provided by passing catalyst from the regeneration zone to the OCP reaction zone, because the regeneration reaction which oxidizes the carbonaceous deposits from the loaded catalyst is exothermic.

Returning to FIG. 1, reaction effluent stream 40 from the reaction zone 210 comprises oxygenate, olefin, water and carbonyl compound comprising formaldehyde and one or both of C2+ aldehyde and ketone. The reaction effluent stream 40 can be passed to an effluent separation zone 220, such as a gas/liquid contactor, particularly a column comprising packing and/or trays, where it is treated with an aqueous liquid stream 285 to provide a water rich stream 215 comprising oxygenate, formaldehyde and water and a water depleted effluent stream 50 comprising olefin and carbonyl compound comprising one or both of C2+ aldehyde and ketone. Typically, the reaction effluent stream 40 is contacted with the aqueous liquid stream 285 such as a water stream, more particularly a cooled aqueous liquid stream such as a cooled water stream, for instance in a quench column. The aqueous liquid stream 285 can condense water from the reaction effluent stream 40 and absorb oxygenate and formaldehyde to provide the water rich stream 215.

The aqueous liquid stream 285 and water rich stream 215 may be part of an effluent separation circuit 275. At least a portion of the water rich stream 215 may be returned to the effluent separation zone 220 to remove oxygenate, formaldehyde and water from the reaction effluent stream 40, for instance as the aqueous liquid stream 285. In such an embodiment, the aqueous liquid stream 285 would further comprise oxygenate and formaldehyde. The embodiment of FIG. 1 shows water rich stream 215 being split into return water rich stream 215a to be passed back to the effluent separation zone and continuing water rich stream 215b.

The water rich stream 215, or continuing water rich stream 215b may then be passed to an oxygenate recovery zone 260, such as an oxygenate stripper. The oxygenate recovery zone 260 may be a column comprising one or both of trays and packing in which the water rich stream 215 is contacted with a stripping gas, for instance a heated aqueous stream such as a steam stream (not shown) to vaporise the oxygenate and formaldehyde. The oxygenate recovery zone can separate oxygenate from the water in the water rich stream to provide a recovered oxygenate stream 225 comprising oxygenate and formaldehyde and an aqueous recovered stream 265 comprising water. The recovered oxygenate stream 225 can be passed to the OTO reaction zone 210 as an oxygenate co-feed stream.

In an embodiment not shown in FIG. 1, the recovered oxygenate stream 225 can be passed to an oxygenate recovered stream heating furnace prior to entering the OTO reaction zone 210 in order to pre-heat the stream to a temperature suitable for use as an oxygenate co-feed.

At least a portion of the aqueous recovered stream 265, such as return aqueous recovered stream 265a, can be passed back to the effluent separation zone 220.

In the embodiment of FIG. 1, the effluent separation circuit comprises at least aqueous liquid stream 285 and water rich stream 215. When a continuous circuit is provided, the effluent separation circuit may further comprise one or more of return water rich stream 215a, continuing water rich stream 215b, aqueous recovered stream 265 and return aqueous recovered stream 265a. Stream 265b may be purged from the process. There should be no transfer of absorbent between the effluent separation circuit and the carbonyl compound absorbent circuit 305 discussed below. This prevents the contamination of the aqueous liquid in the effluent separation circuit 275 with bisulphite and/or carbonyl compound adduct. It will therefore be apparent that the aqueous liquid stream 285 and water rich stream 215, and the streams derived therefrom, should not comprise an aqueous solution of bisulphite having a pH in the range of from 4 to 8 and/or carbonyl compound adduct comprising one or both of C2+ aldehyde adduct and ketone adduct

The water depleted effluent stream 50 comprising olefin and carbonyl compound comprising one or both of C2+ aldehyde and ketone can be passed to an effluent compressor 280, in which the pressure of the stream is increased to provide a compressed effluent stream 55 comprising olefin and carbonyl compound comprising one or both of C2+ aldehyde and ketone. The effluent compressor 280 may be a single stage or a multi-stage compressor. The effluent compressor 280 may be driven by an effluent compressor driver 290, such as an electric motor or a turbine, particularly a steam turbine. The compressed effluent stream 55 may be provided at a pressure above 2.5 bara, typically above 5 bara, more typically above 10 bara. Gas-liquid separators (not shown), such as a knock-out drums, for the removal of any condensed phase such as water and C5+ hydrocarbons may be present after compression, or after each stage of compression if a multi-stage compression system is used.

Subsequently, the compressed effluent stream 55 comprising olefin and carbonyl compound comprising one or both of C2+ aldehyde and ketone can then be passed to a carbonyl compound absorption zone 230. The compressed effluent stream 55 is treated, typically contacted, with a carbonyl compound absorbent stream 255 comprising an aqueous solution of bisulphite having a pH in the range of from 4 to 8. The bisulphite may be present in the aqueous solution as a dissolved salt, such as the salt of an alkali or alkaline earth metal, particularly lithium, sodium and potassium. The bisulphite solution may have a concentration in the range of from 1 to 10 wt. %, more typically 1 to 5 wt. %.

Preferably, the compressed effluent stream 55 is treated with a carbonyl compound absorbent stream 255 at a temperature in the range of from 30 to 50° C., more preferably of from 35 to 45° C.

Bisulphite can react with aldehyde and ketone in the compressed effluent stream 55 to provide an aqueous soluble bisulphite adduct thereby providing an olefinic product stream 70 comprising olefin and a loaded carbonyl compound absorbent stream 235 comprising one or both of C2+ aldehyde adduct and ketone adduct and optionally unreacted bisulphite.

The carbonyl compound absorbent stream 255 and loaded carbonyl compound absorbent stream 235 may be part of an carbonyl compound absorbent circuit 305.

If loaded carbonyl compound absorbent stream 235 comprises unreacted bisulphite, at least a portion of the loaded carbonyl compound absorbent stream 235 may be returned to the carbonyl compound absorption zone 230 to remove carbonyl compound comprising one or both of C2+ aldehyde and ketone from the compressed effluent stream 55, for instance as the carbonyl compound absorbent stream 255. In such an embodiment, the carbonyl compound absorbent stream 255 would further comprise one or both of C2+ aldehyde adduct and ketone adduct. The embodiment of FIG. 1 shows loaded carbonyl compound absorbent stream 235 being split into return loaded carbonyl compound absorbent stream 235a to be passed back to the carbonyl compound absorption zone 230 and continuing loaded carbonyl compound absorbent stream 235b.

In order to replenish the bisulphite in the carbonyl compound absorbent stream 255, a carbonyl compound absorbent restoration stream 205 comprising an aqueous solution of bisulphite can be added to the return loaded carbonyl compound absorbent stream 235a. The concentration of bisulphite in the carbonyl compound absorbent restoration stream 205 may be selected in order provide a desired concentration of absorbent in the carbonyl compound absorbent stream 255. Typically, the carbonyl compound absorbent restoration stream 205 comprises an aqueous solution of bisulphite having a pH in the range of from 4 to 8.

Stream 235b may be purged from the process.

In the embodiment of FIG. 1, the carbonyl compound absorbent circuit 305 comprises at least carbonyl compound absorbent stream 255 and loaded carbonyl compound absorbent stream 235. The carbonyl compound absorbent circuit 305 should be separated from the effluent separation circuit 275 as already discussed.

The olefinic product stream 70 comprising olefin may then be further treated. If the reaction effluent stream 40 further comprises an acid gas which has not already been removed, the olefinic product stream 70 may be treated to remove any acid gas such as hydrogen sulphide or carbon dioxide present. For instance, the olefinic product stream 70 may be contacted with an acid gas absorbent stream, such as an aqueous alkaline stream, typically a stream comprising an alkali metal hydroxide, to absorb the acid-forming gas. This embodiment is not shown in FIG. 1 but is discussed in relation to the embodiments of FIGS. 2 and 3.

The olefinic product stream 70, which has optionally been treated to remove acid gas, may then be optionally compressed in an olefinic product compressor 310 to provide a compressed olefinic product stream 75 comprising olefin. The olefinic product compressor 310 may be a single or multi-stage compressor, driven by an olefinic product compressor driver 320, such as an electric motor or turbine, particularly a steam turbine. Olefinic product compressor 310 and effluent compressor 280 may share a common driver. Typically, any compression would provide the compressed olefinic product stream 75 at a pressure of above 25 bara, more typically in the range of from 30 to 40 bara. The olefinic product compressor 310 may optionally further comprise gas/liquid separators, such as knock-out drums, to remove any condensed phase produced.

The optionally compressed olefinic product streams 75 may be passed to an olefinic separation zone 250, such as a distillation zone, preferably a cryogenic distillation zone, to provide two or more olefinic component streams 120, 130, 140.

The olefinic product preferably comprises two or more of the group selected from ethylene, propylene, butylene(s) and pentylene(s). Consequently, each of the two or more olefinic component streams may comprise at least one of the group selected from ethylene, propylene, butylene(s) and pentylene(s). In the embodiment of FIG. 1, olefinic separation zone 250 may comprise a deethaniser providing a first olefinic component stream 120 comprising ethylene, a depropaniser providing a second olefinic component stream 130 comprising propylene and a third olefinic component stream 140 comprising C4+ hydrocarbons including C4+ olefins such as one or more of butylene (s) and pentylene(s).

In an embodiment not shown in FIG. 1, at least a portion of the third olefinic component stream 140 can be passed to the OTO reaction zone 210 as olefinic co-feed stream 20.

FIGS. 2 and 3 disclose further aspects of the process and apparatus disclosed herein, in which the compressed effluent stream 55 is also treated to remove acid gas.

The apparatus of the embodiment of FIG. 2 can be used in the process and apparatus of the embodiment of FIG. 1. Apparatus and stream lines of the same reference numerals to that of FIG. 1 correspond to identical equipment.

Reaction effluent stream 40, and therefore compressed effluent stream 55, may both further comprise acid gas such as carbon dioxide, which can be produced in the OTO reaction zone (FIG. 1, 210) by side reactions of the OTO process. Other acid gases, such as hydrogen sulphide are not commonly produced by an OTO process, but may be present in the compressed effluent stream 55, for instance if this is supplemented with effluent from another source, such as a naphtha cracker.

FIG. 2 discloses an embodiment in which a compressed effluent stream 55 further comprising acid gas such as carbon dioxide is passed to a carbonyl compound absorption zone 230 to provide an olefinic product stream 70 comprising olefin and acid gas. The carbonyl compound absorbent circuit 305 operates in a similar manner to the embodiment of FIG. 1, with a portion of the loaded carbonyl compound absorbent stream 235 being returned to the carbonyl compound absorption zone 230 via return loaded carbonyl compound absorbent stream 235a with the remaining portion drawn off as continuing loaded carbonyl compound absorbent stream 235b. Additional aqueous bisulphite solution is added to the return loaded carbonyl compound absorbent stream 235a as carbonyl compound restoration stream 205 to provide a carbonyl compound absorbent stream 255. The olefinic product stream 70 comprising olefin and acid gas can then be passed to an acid gas absorption zone 420, in which it is treated with an acid gas absorbent stream 415 comprising acid gas absorbent, said stream in liquid form, to provide an acid gas depleted olefinic product stream 70a comprising olefin. The acid gas absorption zone 420 may be a column optionally comprising one or both of packing and trays. The acid gas absorbent stream 415 may be an aqueous alkaline stream, such as an aqueous sodium hydroxide stream, particularly comprising 2 to 10 wt. % sodium hydroxide. Alternatively, there are two separate sodium hydroxide circuits, e.g. one having a concentration of 2 to 5% and the one before the water wash of 5 to 10%.

Absorption of acid gas by the acid gas absorbent stream 415 provides a loaded acid gas absorbent stream 425 comprising acid gas absorbent and acid gas, said stream in liquid form. A portion of the loaded acid gas absorbent stream 425 can be returned to the acid gas absorption zone 420 via return loaded acid gas stream 425a, with the remainder forming continuing loaded acid gas absorbent stream 425b. Additional acid gas absorbent can be added to the return loaded acid gas absorbent stream 425a as acid gas restoration stream 435 to provide a acid gas absorbent stream 415.

Acid gas depleted olefinic product stream 70a may further comprise components from one or both of the absorbent streams such as acid gas absorbent and loaded acid gas absorbent. Absorbent entrained in the acid gas depleted olefinic product stream 70a may be removed by treatment with an aqueous wash stream 455, such as a water stream, in a wash zone 440. Wash zone 440 may be a wash column, which may comprise one or both of trays and packing. Wash zone 440 provides a spent aqueous wash stream 465 and a washed acid gas depleted olefinic product stream 70b. The spent aqueous wash stream 465 may comprise water acid gas absorbent and loaded acid gas absorbent. The washed acid gas depleted olefinic product stream 70b, which comprises olefin, may be depleted, compared to the acid gas depleted olefinic product stream 70a, in one or more of the group comprising acid gas absorbent and loaded acid gas absorbent.

The washed acid gas depleted olefinic product stream 70b may be optionally dried, before optional compression in the olefinic product compressor 310 and further treatment as discussed in the embodiment of FIG. 1.

FIG. 3 shows an embodiment in which the carbonyl compound absorption zone and acid gas absorption zone are provided in the same shell. Apparatus and stream lines having the same reference numerals to those of FIGS. 1 and 2 represent identical equipment. The embodiment of FIG. 3 can be used in conjunction with the process and apparatus of FIG. 1.

Compressed effluent stream 55 further comprising acid gas such as carbon dioxide or hydrogen sulphide can be passed to a combined carbonyl compound and acid gas absorption column 500. The gravitationally lowest zone is the carbonyl compound removal zone in which the compressed effluent stream is treated with a carbonyl compound absorption stream 255 to provide an olefinic product stream and a loaded carbonyl compound absorbent stream 235.

The olefinic product stream comprising olefin and acid gas may then be treated in a plurality of acid gas absorption zones, which can be located gravitationally higher than the carbonyl compound removal zone. The acid gas absorption zones can treat the olefinic product stream with a plurality of acid gas absorbent streams 415a, 415b, each of increasing concentration of acid gas absorbent. The gravitationally higher the acid gas absorption zone, the higher the concentration of the acid gas absorbent in the acid gas absorption stream 415a, 415b.

For instance, the olefinic product stream may be treated with a first acid gas absorbent stream 415a, such as an aqueous sodium hydroxide stream having a concentration of approximately 2 wt. % sodium hydroxide, and subsequently treated with a second acid gas absorbent stream 415b, such as an aqueous sodium hydroxide stream having a concentration of approximately 6 wt. % sodium hydroxide. The plurality of acid gas absorption zones can be in fluid communication such that a single loaded acid gas absorption stream 425 exits column 500. The acid gas removal zones can provide an acid gas depleted olefinic product stream, which can then be passed to a wash zone, gravitationally higher than the acid gas removal zones, in which it is treated with an aqueous wash stream 455 to provide washed acid gas depleted olefinic product stream 70a and spent wash stream 465.

The person skilled in the art will understand that the present invention can be carried out in many various ways without departing from the scope of the appended claims.

1. A process for the preparation of olefinic product, the process comprising at least the steps of:
reacting an oxygenate feedstock comprising oxygenate in an oxygenate reaction zone in the presence of a catalyst comprising a molecular sieve to produce a reaction effluent stream comprising oxygenate, olefin, water and carbonyl compound comprising formaldehyde and one or both of C2+ aldehyde and ketone;
treating the reaction effluent stream with an aqueous liquid stream to provide a water rich stream comprising oxygenate, formaldehyde and water and a water depleted effluent stream comprising olefin and carbonyl compound comprising one or both of C2+ aldehyde and ketone, said aqueous liquid stream and said water rich stream present in an effluent separation circuit;
compressing the water depleted effluent stream, with the optional removal of any condensed phase, to provide a compressed effluent stream; and
treating the compressed effluent stream with a carbonyl compound absorbent stream comprising an aqueous solution of bisulphite having a pH in the range of from 4 to 8, to provide an olefinic product stream comprising olefin and a loaded carbonyl compound absorbent stream comprising an aqueous solution of at least one carbonyl adduct comprising one or both of C2+ aldehyde adduct and ketone adduct and optionally unreacted bisulphite, said carbonyl compound absorbent stream and said loaded carbonyl compound absorbent stream in a carbonyl compound absorbent circuit separate from the effluent separation circuit.

2. The process of claim 1, further comprising the steps of:
separating at least a part of the water rich stream into an oxygenate recovered stream comprising oxygenate and an aqueous recovered stream comprising water; and
passing the oxygenate recovered stream to the oxygenate reaction zone.

3. The process of claim 1 further comprising the step of:
passing at least a part of the water rich stream to the aqueous liquid stream.

4. The process of claim 1 further comprising the step of:
passing at least a part of the loaded carbonyl compound absorbent stream to the carbonyl compound absorbent stream.

5. The process of claim 1 further comprising the steps of:
removing at least a portion of the loaded carbonyl compound absorbent stream as a continuing loaded carbonyl compound absorbent stream; and
adding carbonyl compound absorbent comprising an aqueous solution of bisulphite having a pH in the range of from 4 to 8 to the carbonyl compound absorbent stream as a carbonyl compound absorbent restoration stream.

6. The process of claim 1, in which the pH of the aqueous solution of bisulphite is in the range of from 5 to 7.

7. The process of claim 1, wherein the C2+ aldehyde comprises one or both of a C2+ saturated aldehyde, such as acetaldehyde.

8. The process of claim 1, wherein the reaction effluent stream, water depleted stream, compressed effluent stream and olefinic product stream each further comprise an acid gas, such as one or both of carbon dioxide and hydrogen sulphide, and said process further comprises the step of:
treating the olefinic product stream with an acid gas absorbent stream to provide a loaded acid gas absorbent stream and an acid gas depleted olefinic product stream comprising olefin.

9. The process of claim 8 wherein the acid gas depleted olefinic product stream comprises two or more of the group selected from ethylene, propylene, butylenes, pentylenes and hexylenes, said process further comprising the steps of:
drying and optionally compressing the acid gas depleted olefinic product stream to provide a dried acid gas depleted olefinic product stream; and
separating the dried acid gas depleted olefinic product stream into two or more olefinic component streams, each said olefinic component stream comprising at least one of the group selected from ethylene, propylene, butylenes, pentylenes and hexylenes.

10. The process of claim 1, wherein the oxygenic feedstock is reacted to produce the reaction effluent stream in the presence of an olefinic co-feed, such as an olefinic co-feed comprising one or both of butylene and pentylene.

11. The process of claim 1 wherein the molecular sieve is selected from the group comprising silicoaluminophosphate and aluminosilicate.

12. The process of claim 11 wherein the molecular sieve is an aluminosilicate having a 10-membered ring zeolite structure.

13. The process of claim 11 wherein the aluminosilicate comprises one or more of the group comprising a TON-type aluminosilicate, such as ZSM-22, a MTT-type aluminosilicate, such as ZSM-23, MEL-type aluminosilicate, such as ZSM-11 and MFI-type aluminosilicate, such as ZSM-5.

14. An apparatus for the preparation of an olefinic product, from an oxygenate feedstock, said apparatus comprising at least:
an oxygenate reaction zone comprising a catalyst comprising molecular sieve, said oxygenate reaction zone having a first inlet for an oxygenate feedstock stream comprising oxygenate and a first outlet for a reaction effluent stream comprising oxygenate, olefin, water and carbonyl compound comprising formaldehyde and one or both of C2+ aldehyde and ketone, said first outlet in fluid communication with a first inlet of an effluent separation zone;
an effluent separation zone for separating the reaction effluent stream into a water rich stream comprising oxygenate, formaldehyde and water and a water depleted effluent stream comprising olefin and carbonyl compound comprising C2+ aldehyde and ketone, said effluent separation zone having a first inlet for the reaction effluent stream, a second inlet for an aqueous liquid stream, a first outlet for the water rich stream and a second outlet for the water depleted effluent stream, said second outlet in fluid communication with the inlet of an effluent compressor, wherein said aqueous liquid stream and said water rich stream comprise an effluent separation circuit;
an effluent compressor having a first inlet for the water depleted effluent stream and a first outlet for a compressed effluent stream, said first outlet in fluid communication with the first inlet of an carbonyl compound absorption zone, said effluent compressor optionally comprising gas/liquid separation means for the removal of any condensed phase; and
a carbonyl compound absorption zone having a first inlet for the compressed effluent stream, a second inlet for a carbonyl compound absorbent stream comprising an aqueous solution of bisulphite having a pH in the range of from 4 to 8, a first outlet for an olefinic product stream comprising olefin and a second outlet for a loaded carbonyl compound absorbent stream comprising an aqueous solution of at least one carbonyl compound adduct comprising one or both of C2+ aldehyde adduct and ketone adduct and optionally unreacted bisulphite, wherein said carbonyl compound absorbent stream and said loaded carbonyl compound absorbent stream form a carbonyl compound absorbent circuit, said carbonyl compound absorbent circuit being separate from the effluent separation circuit.
